(19) United States
(12) Patent Application Publication
(10) Pub. No.: US 2013/0280346 A1
(43) Pub. Date: Oct. 24, 2013

(54) Infusates with Enhanced pH Stability Under Ethylene Oxide Sterilization

(71) Applicant: C. R. BARD, INC., Murray Hill, NJ (US)

(72) Inventors: Kelly B. Powers, North Salt Lake, UT (US); Murtaza Yusuf Amin, Farmington, UT (US); Matthew R. Trebella, Stansbury Park, UT (US); James L. Freasier, Tooele, UT (US); Raymond Kerns, West Valley City, UT (US)

(73) Assignee: C. R. BARD, INC., Murray Hill, NJ (US)

(21) Appl. No.: 13/866,960

(22) Filed: Apr. 19, 2013

Related U.S. Application Data
(60) Provisional application No. 61/635,654, filed on Apr. 19, 2012.
(60) Provisional application No. 61/785,175, filed on Mar. 14, 2013.

(51) Int. Cl. A61L 2/20

(52) U.S. Cl. CPC A61L 2/206
USPC 424/663

(57) ABSTRACT

Normal saline and other infusate solutions for infusion into the body of a patient during medical treatment are disclosed. In particular, infusates are disclosed that are formulated to resist changes to the pH of the solution when subjected to sterilization procedures that employ ethylene oxide gas. In one embodiment, a buffered infusate suitable for disposal in a syringe or other container is disclosed. The syringe is sterilizable using ethylene oxide. The buffered infusate comprises an aqueous solution that is disposed in the syringe and is suitable for infusion into a body of a patient. A buffer component is added to the aqueous solution to form a buffered solution. The buffer component is configured to resist a change in the pH of the buffered solution upon exposure of the buffered solution to the ethylene oxide during sterilization of the syringe.

### CROSS-REFERENCE TO RELATED APPLICATIONS

This application claims the priority benefit of U.S. Provisional Application No. 61/635,654, filed Apr. 19, 2012, titled “Saline Solution PH-Stable Under Ethylene Oxide Sterilization;” and U.S. Provisional Application No. 61/785,175, filed Mar. 14, 2013, titled “Saline Solution PH-Stable Under Ethylene Oxide Sterilization,” each of which applications is incorporated herein by reference in its entirety.

### BRIEF SUMMARY

Briefly summarized, embodiments of the present invention are directed to normal saline and other infusate aqueous solutions for infusion into the body of a patient during medical treatment. In particular, infusates are disclosed that are formulated to resist changes to the pH of the solution when subjected to sterilization procedures that employ ethylene oxide (“EO”) gas, also referred to herein as EO sterilization. EO sterilization is a common method for sterilizing various medical devices and components. When infusates are disposed in containers that are sterilized via EO sterilization, any permeation of the EO gas into the device so as to interact with aqueous solution of the infusate can undesirably alter the pH of the solution. An example of such a device containing an infusate where EO gas permeation can alter the infusate pH includes a syringe used for dispensing a saline solution into a catheter inserted into the body of a patient, for instance.

In one embodiment, a buffered infusate suitable for disposal in a syringe or other container is disclosed. The syringe itself is sterilizable using ethylene oxide. The buffered infusate comprises an aqueous solution, such as saline, which is disposed in the syringe and is suitable for infusion into a body of a patient. A buffer component is added to the saline solution to form a buffered saline solution. The buffer component is configured to resist a change in the pH of the buffered saline solution upon exposure of the buffered saline solution to the ethylene oxide during sterilization of the syringe.

In one embodiment, the buffer component includes an acid and conjugate base pair, such as acetic acid and sodium acetate. However, as is discussed below, many other substances can be included in the buffer component.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 is a perspective view of a catheter assembly and a syringe attached thereto, serving as one example environment wherein an embodiment of the present disclosure can be practiced;

FIG. 2 is a perspective view of the syringe of FIG. 1, according to one embodiment; and

FIG. 3 shows a process for producing a fluid-filled syringe according to one embodiment.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a catheter placed within the body of a patient is considered a distal end of the catheter, while the catheter end remaining outside the body is a proximal end of the catheter. Also, the words “including,” “has,” and “having,” as used herein, including the claims, shall have the same meaning as the word “comprising.”

Embodiments of the present invention are generally directed to infusates, that is, solutions for infusion into the body of a patient during medical treatment. Saline and other aqueous solutions are examples of such infusates. In particular, infusates are disclosed herein that are formulated to resist changes to the pH of the solution when subjected to sterilization procedures that employ ethylene oxide (“EO”) gas, also referred to herein as EO sterilization. EO sterilization, when used to sterilize syringes or other medical containers in which infusates such as saline solutions are disposed, can undesirably alter the pH of the solution via interaction of the EO gas with the aqueous solution of the infusate.

In further detail, it is noted that syringes and other containers pre-filled with saline solution (also referred to herein as saline), for instance, are popular with medical clinicians for inclusion in various medical device and procedure kits as they offer enhanced convenience for the clinician. For instance, syringes are employed to flush saline through an indwelling catheter, such as a PICC. Saline is also employed in flushing implanted port-catheter assemblies and in other applications. The saline solution in such syringes is restricted by United States Pharmacopeia (“USP”) guidelines to possess a pH between 4.5 and 7.0 in order to be suitable for human use. In addition, USP requirements also restrict the osmolarity, sodium ion level, and chlorine ion level in the saline solution to within specified ranges.

Because the above-described pre-filled syringes are typically manufactured from plastic, such as polypropylene, certain complications arise. Chief among these complications concerns the manner in which the syringes are sterilized. Generally, one preferred manner for sterilization of medical devices is via EO gas. Indeed, many of the kits mentioned above are sterilized via EO sterilization. However, it has been shown in one example that the pH of saline contained by plastic pre-filled syringes, when the syringes are subjected to EO sterilization, increases from a pH of about 5 before EO sterilization to about 9 after EO sterilization due to permeation of EO into the saline solution through one or more routes into the plastic syringe. Equation (1) below shows that interaction of the EO gas with components of the saline solution produces chlorohydrin:

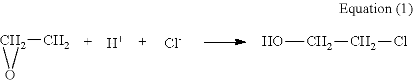

Note that the EO sterilization process is employed to sterilize the syringe itself, and not the infusate contained therein. The infusate can be sterilized by other methods, including heat sterilization, such as via autoclaving (steam sterilization), gamma sterilization, starting with sterile components and maintain the sterility during filling into the container, etc.

The above reaction shown in Equation 1 consumes an H+ hydrogen ion, resulting in a net increase of pH in the solution. Thus, EO sterilization of saline-filled plastic syringes typically and undesirably increases the pH of the saline inside such that it no longer conforms to USP guidelines. The same process can undesirably affect other infusates and also affect syringes made from other materials in addition to plastic.

As a result, it often becomes necessary to omit plastic pre-filled saline syringes from medical kits during EO sterilization to avoid undesirably altering the pH of the saline. Instead, the syringes are sterilized by an alternate process, such as via steam or gamma beam sterilization, then attached to the kit post-sterilization in a separate container, often called a sidecar, before shipment to the customer. This involves additional processing and packaging steps, increases overall kit cost, and represents an inconvenience for kit users. The ability to include plastic saline-filled syringes directly in a medical kit prior to EO sterilization without unacceptably altering the saline pH would represent a significant savings in terms of manufacturing efficiency, time, and cost.

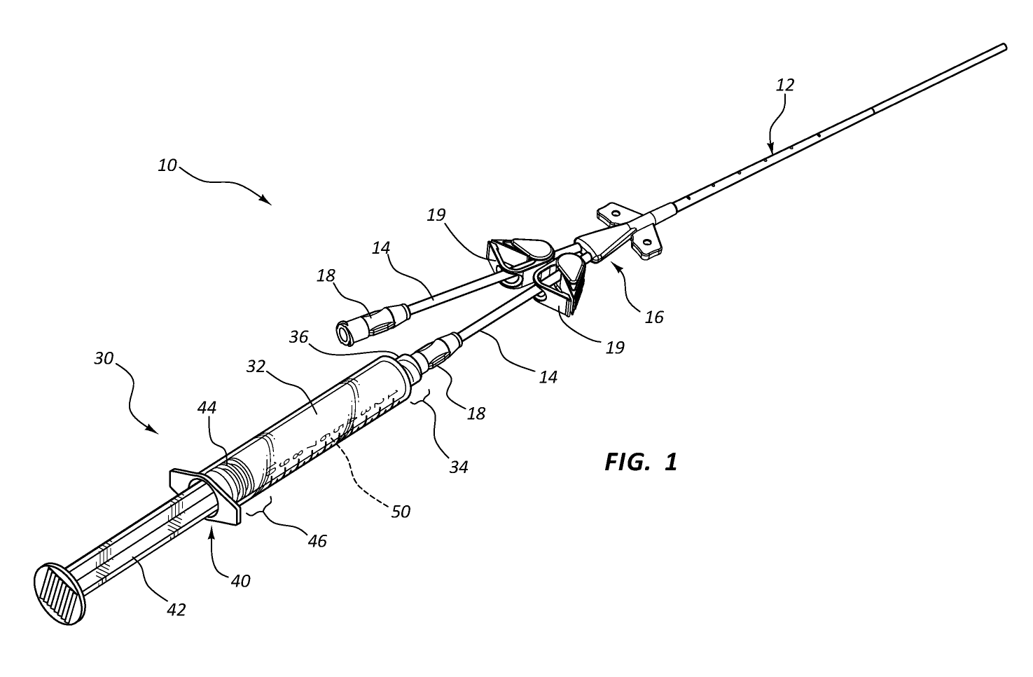

In light of the above, reference is made to FIG. 1, which shows a catheter assembly 10 (“catheter”). The catheter 10 includes an elongate catheter tube 12 defining one, two, or more lumens. As shown here, the catheter 10 includes two extension legs 14 that are each fluidly connected to one of two lumens of the catheter tube 12 via a bifurcation 16. A female or other suitable type of luer connector 18 is included on a proximal end of each of the extension legs 14. Clamps 19 are also included on the extension legs 14 to selectively impede fluid flow therethrough.

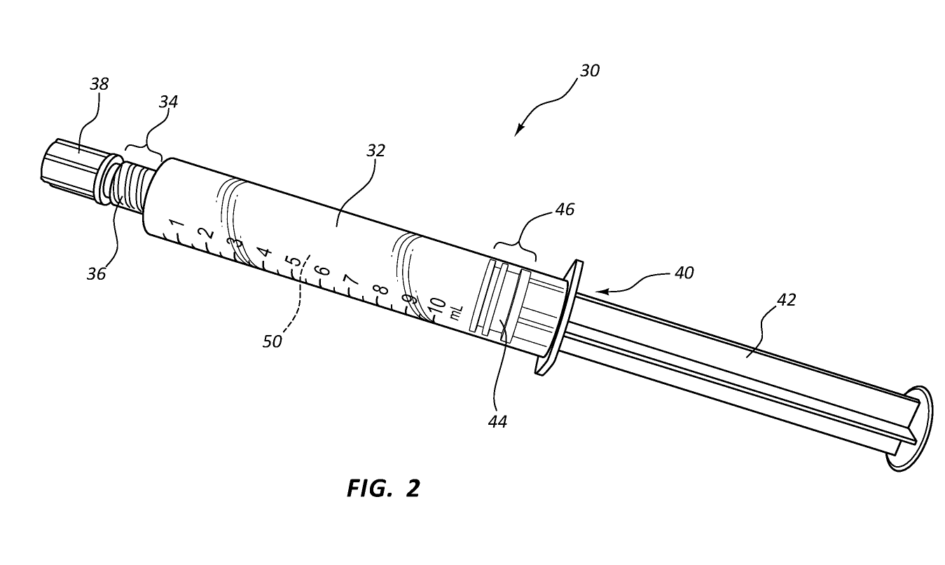

FIG. 1 further shows a syringe 30 operably attached to one of the extension legs 14 via the corresponding luer connector 18. As shown, the syringe 30 includes a plastic, barrel-shaped, hollow body 32 and a tip portion 34 that defines a male, threaded fluid port 36. An end cap 38 (FIG. 2) can be threadably engaged with the tip portion 34 to prevent fluid escape through the fluid port 36.

The syringe 30 further includes a plunger 40 that in turn includes a plunger rod 42 and a plunger tip 44 disposed at a distal end of the plunger rod and disposed within the syringe body 32. The plunger tip 44 includes one or more septa 46 that each form a relatively tight but slidable fit with the inner wall of the hollow body 32. Here, three septa 46 are included on the plunger tip 44.

The hollow body 32 of the syringe 30 is pre-filled with an aqueous solution containing sodium chloride, that is, a saline solution. As mentioned, the syringe 30 is operably connected to the catheter 10 via threaded engagement of the fluid port 36 with the luer connector 18 of a corresponding one of the extension legs 14. The plunger rod 42 can be pushed distally into the syringe body 32 in order to cause the plunger tip 44 to force the saline out the fluid port 36 and into the corresponding extension leg 14 for delivery to the lumen of the catheter tube 12. In this way, flushing with saline of one or more lumens of the catheter tube 12 can occur. As will be described, in accordance with present embodiments, the pH of the saline solution within the syringe desirably remains within an acceptable pH range after EO sterilization, thus rendering it acceptable for use in patient infusion, catheter and port flushing, and other medical procedures. Note that, though directed to syringes, the principles described herein can be applied to other infusate-holding containers, including ampoules and the like. Note also that, though the discussion herein relates to normal saline solutions of a predetermined saline concentration, saline solutions with concentrations other than normal are contemplated. Also, other aqueous solutions can also benefit from the principles described herein.

In accordance with one embodiment, the pH-increasing effects of EO sterilization on saline solution contained in plastic syringes can be counteracted by providing a saline solution that can resists such effects. In one embodiment, this is achieved by the inclusion of a suitable buffer component in predetermined quantity to the saline or other suitable aqueous solution prior to EO sterilization. In one embodiment, a normal saline solution, i.e., 0.9% w/w sodium chloride (“NaCl”) in water solution) is buffered with a suitable buffer component before the solution is inserted into the plastic syringe. The addition of the buffer component to the saline solution desirably inhibits the increase in pH of the saline solution as a result of EO gas permeation into the saline solution during EO sterilization. Thus, the pH of the saline solution in the plastic syringe remains in the 4.5-7 range required by the USP guidelines, even after EO sterilization. This in turn enables the saline syringe to be originally included in a kit, such as a PICC catheter insertion kit for instance, and be process with EO sterilization along with the other kit components. As mentioned, this saves on kit manufacturing costs, manufacturing efficiency, and customer convenience while preserving the quality of the saline solution. Indeed, the inclusion of the syringe within a sterile kit enables the syringe to be pulled directly from the kit by a clinician within the sterile field itself during a medical procedure, as opposed to being removed from the sidecar—the sidecar having a non-sterile exterior—and introduced into the sterile field by another person. Note that further details regarding EO sterilization are found further below.

In one embodiment, the buffer component that is added to the saline solution includes an acid and its conjugate base. In the present embodiment, an acetate-based combination is used, including acetic acid and its conjugate base, sodium acetate. These two sub-components (which may be in solid or liquid form) are added, in one embodiment, in predetermined quantities to a solid or liquid-state sodium chloride sub-component during manufacture of the saline solution. Water, such as purified, deionized water, is then added to the admixture to produce the proper saline solution concentration. The resultant buffered solution exhibits the desired pH change-resisting characteristics described above.

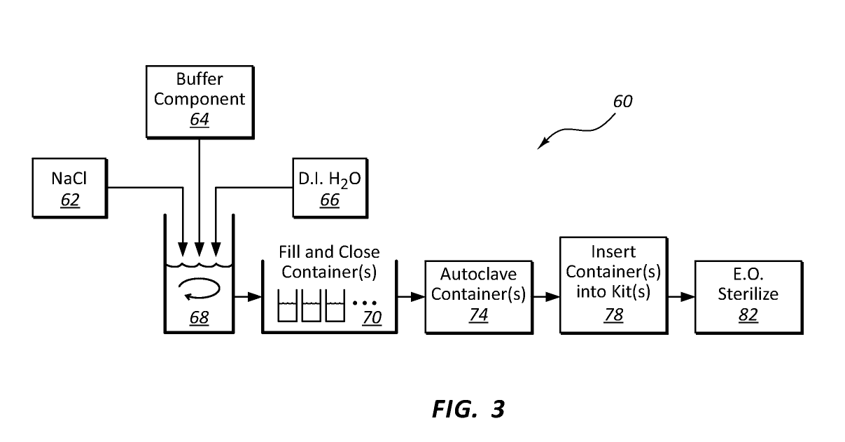

In further detail, FIG. 3 generally describes a process 60 for providing and sterilizing a buffered saline solution. A predetermined amount of sodium chloride 62 is combined with a predetermined quantity of a buffer component 64 and deionized water 66. In the case where the buffer component 64 includes acetic acid and its conjugate base of sodium acetate, in one embodiment the sodium chloride 62 in crystal form is dry-mixed with a powder form of sodium acetate. These mixed components can then be mixed with the acetic acid, in liquid form, in a vessel 68 before the water 66 is added in the vessel to form a buffered saline solution and bring it to the desired liquid volume.

At stage 70 the buffered saline solution is filled into one or more syringes, such as the syringe 30 shown in FIGS. 1 and 2, or other suitable container(s). In the case of syringes, the plungers and end caps of each syringe are attached to the syringe body after filling. At stage 74, the syringes—each filled with the buffered saline solution—are heat sterilized, such as via autoclaving, or otherwise treated to sterilize the saline solution itself, if desired. Instead of heat sterilizing, in one embodiment the buffered saline solution can be manufactured in a sterile environment using sterile components, with sterility being maintained through filling of the solution into the containers.

After the heat sterilization, the syringes at stage 78 are inserted into one or more packages, such as medical kits, including catheter kits, port kits, etc. Such kits are typically sealed with plastic or other suitable barrier. At stage 82, the kits are EO sterilized with the use of EO gas, which sterilizes the kit components, including the external portions of the syringes themselves.

To the extent that EO gas has permeated the syringe and interacted with the buffered saline solution contained therein, the saline solution is subject to the effects of the EO gas, including the production of ethylene chlorohydrin and the corresponding loss of hydrogen ions in the solution, resulting in a rise in solution pH. However, the presence in the saline solution of the buffer component causes the acetic acid and acetate base constituents of the buffer component to work in mitigating the increase in pH caused by the creation of the ethylene chlorohydrins. The effectiveness of the buffer component in preventing pH change brought on by the ethylene chlorohydrin is dependent upon the amount of buffer component present in the buffered saline and the amount of ethylene chlorohydrin produced, but the buffer component is operative in present embodiments in resisting the pH change, which can assist the saline to remain within the USP pH guidelines discussed above. Note that the osmolarity, sodium ion level, and chlorine ion level in the buffered saline solution can also be maintained within USP requirements post-EO sterilization according to present embodiments.

Below is an example preparation of a buffered normal saline solution, together with post EO sterilization pH effects, in accordance with one embodiment.

### EXAMPLE 1

A buffered normal saline solution including a 0.0100M acetate buffer component was prepared by adding together and mixing the components listed in Table (1) below in the noted amounts/concentrations with enough ultrapure, deionized water to produce one liter of solution:

TABLE (1)
Acetate-Buffered Normal Saline Formulation

| | | Solid | Acetic Acid |
|---|---|---|---|
| | Solid Sodium | Sodium | (g of 1N solution |
| | Chloride | Acetate | or mL of 1 Molar |
| Solution Characteristics | (g per L) | (g per L) | solution per L) |
| 0.0100 Molar Acetate, | 8.730 | 0.683 | 1.669 |
| pH = 5.25 | | | |

After preparation according to the above formulation, the buffered normal saline solution was predicted to exhibit the solution characteristics shown in Table (2), below:

TABLE (2)
Acetate-Buffered Normal Saline Solution Characteristics

| | [Na⁺] | [Na⁺]/ | [Cl⁻] | [Cl⁻]/ | Osmolarity | Osmolarity/ |
|---|---|---|---|---|---|---|
| Solution | (mol/L) | [Na⁺ₙₒₘᵢₙₐₗ] | (mol/L) | [Cl⁻ₙₒₘᵢₙₐₗ] | (mmol/L) | Nominal |
| 0.0100M | 0.158 | 1.024 | 0.149 | 0.970 | 319 | 1.03 |
| Acetate | | | | | | |
| pH = 5.25 | | | | | | |

Nominal here refers to the concentration found in 0.9% normal saline without acetate buffer. Na⁺ nominal = 0.154 mol/L, Cl⁻ nominal = 0.154 mol/L, and osmolarity nominal = 309 mMol/L.

The buffered normal saline solution was transferred into syringes, the syringes assembled so that air pockets were substantially removed from the fluid cavities, and the assembled syringes were heat sterilized via autoclave to sterilize the solution within the syringes. The syringes were then subjected to two cycles of EO sterilization, with each cycle exposing the solution-filled syringes to EO gas at a temperature of about 135 degrees F. at about 60% relative humidity at a pressure of about 28 inches of mercury for an EO gas exposure time of at least 2 hours. This process was prefaced and followed by standard pre-conditioning and post-conditioning procedures.

In a formulation example similar to the above, the pH of the buffered normal saline solution was measured at the time of mixing the solution components after syringe filling, and after two cycles of the above-described EO sterilization process. The pH results are shown in Table (3), below:

TABLE (3)
pH Measurement at Various Stages

| | pH | pH after filling | pH after | pH after 2x EO |
|---|---|---|---|---|
| | at Mixing | Syringes | Autoclave | Sterilization |
| | (n = 1) | (n = 5) | (n = 5) | (n = 5) |
| Group 1A | 5.50 | 5.460 ± 0.064 | 5.102 ± 0.108 | 5.698 ± 0.088 |

Note:
Values listed are pH, which have no units. Values are means of the sample size at the top of the column. Standard deviations are given as [±x] after the mean value.

As can be seen from Table (3), the pH of the buffered normal saline solution after EO sterilization rose, but stayed within USP pH requirements (a pH of between 4.5 and 7) for normal saline despite exposure of the solution to EO gas during sterilization. Further, the mean sodium chloride concentration in the buffered normal saline solution was about 0.866%, falling within USP acceptance criteria of between 0.855% and 0.945%.

Note that, in the above example wherein the buffer component includes acetic acid and sodium acetate, the sodium component of the sodium acetate is a cation and serves as a spectator ion in the buffered saline solution. As such, the sodium ion adds no other component than what is already present due to sodium chloride also being present in the solution.

As described above, the acetic acid/sodium acetate acid and conjugate base combination employed for the buffer component in the above example is but one combination that can be employed for the buffer component. Indeed, other acid/conjugate bases can be employed as the buffer component, as appreciated by those skilled in the art. Examples of other acid/conjugate base buffer components include the following:

citric acid/sodium citrate

formic acid/sodium formate

ascorbic acid/sodium ascorbate

lactic acid/sodium lactate

phosphoric acid/sodium phosphate

benzoic acid/sodium benzoate

sorbic acid/sodium sorbate

maleic acid/sodium malate

boric acid/sodium borate

carbonic acid/sodium bicarbonate

In light of the above example pairs, it is appreciated that, generally, weak acids can be paired with their conjugate bases, and weak bases can be paired with their conjugate acids to serve as the buffer component. The above and other suitable acid/conjugate base combinations are therefore contemplated.

Furthermore, other acid/base combinations in addition to the above-described acid/conjugate combinations, can also be employed as the buffer component. For example, a weak acid (an acid with a relatively low degree of dissociation in solution) can be paired with a strong base (a base with a relatively high degree of dissociation in solution) to serve as the buffer component. A buffer component including acetic acid, a weak acid, paired with sodium hydroxide, a strong base, is an example of this. Correspondingly, a weak base (a base with a relatively low degree of dissociation in solution) can be paired with a strong acid (an acid with a relatively high degree of dissociation in solution) to serve as the buffer component. A buffer component including hydrochloric acid, a strong acid, paired with sodium ascorbate, a weak base, is an example of this. Other possible relatively strong acids that could be employed include carbonic acid, phosphoric acid, and nitric acid. Other possible strong bases that could be employed include sodium hydroxide. Bases employed should be biocompatible and sufficiently soluble. These and other combinations are therefore contemplated.

Further to the above, it is appreciated that though the above bases utilize sodium as the cation, in one embodiment other suitable cations can be employed, including potassium, calcium, and magnesium. A cation employed in this manner should be biocompatible, including its safe presence at the resulting concentrations in the bloodstream of a patient, should not form precipitate, and be otherwise compatible with the infusate.

Below are further actual and prophetic examples of preparations of a buffered normal saline solution, in accordance with one embodiment.

### EXAMPLE 2

A buffered normal saline solution including a 0.0100M acetate buffer component was prepared by adding together and mixing the components listed in Table (4) below in the noted amounts/concentrations with enough ultrapure, deionized water to produce one liter of solution:

TABLE (4)

| Component | Amount |
|---|---|
| Sodium Chloride | 8.805 g of solid |
| Acetic Acid | 0.100 g of pure liquid or 1.669 mL of 1M solution |
| Sodium Acetate | 0.683 g of solid |

Bring solution volume to 1 L after dissolving the above solutes

The buffered normal saline solution formulation of Table (4) when prepared possessed the following properties:

TABLE (5)

| | |
|---|---|
| pH | ~5.25 |
| Na⁺ | 0.159 mol/L (1.032 × the nominal of 0.154) |
| Cl⁻ | 0.151 mol/L (0.981 × the nominal of 0.154) |
| Osmolarity | 0.320 mol/L (1.036 × the nominal of 0.309) |
| Total Acetate (as acetic | 0.010 mol/L |
| acid or as acetate) | |

The buffered normal saline solution formulation of Table (4) produced an acetate-based buffer in the saline solution, including sodium ions, acetic acid, and acetate ions, according to the following reaction:

C₂H₄O₂+NaC₂H₃O₂→Na⁺(aq)+C₂H₄O₂(aq)+C₂H₃O₂⁻(aq) Equation (2)

The buffered normal saline solution was then suitable for dispensing, sterilization, and use as has been described elsewhere herein.

### EXAMPLE 3 (PROPHETIC)

A buffered normal saline solution including a 0.0100M acetate buffer component can be prepared by adding together and mixing the components listed in Table (6) below in the noted amounts/concentrations with enough ultrapure, deionized water to produce one liter of solution:

TABLE (6)

| Component | Amount |
|---|---|
| Sodium Chloride | 8.805 g of solid |
| Acetic Acid | 0.600 g of pure liquid or 10.01 mL of 1M solution |
| Sodium Hydroxide | 0.333 g of solid |

Bring solution volume to 1 L after dissolving the above solutes.

The buffered normal saline solution formulation of Table (6) when prepared possesses the following properties:

TABLE (7)

| | |
|---|---|
| pH | ~5.25 |
| Na⁺ | 0.159 mol/L (1.032 × the nominal of 0.154) |
| Cl⁻ | 0.151 mol/L (0.981 × the nominal of 0.154) |
| Osmolarity | 0.320 mol/L (1.036 × the nominal of 0.309) |
| Total Acetate (as acetic | 0.010 mol/L |
| acid or as acetate) | |

The buffered normal saline solution formulation of Table (6) produces an acetate-based buffer in the saline solution according to the following reaction:

C₂H₄O₂+NaOH→C₂H₄O₂(aq)+Na⁺(aq)+OH⁻(aq)⇄H₂O+C₂H₃O₂⁻(aq)+Na⁺(aq) Equation (3)

The buffered normal saline solution can then be suitable for dispensing, sterilization, and use as has been described elsewhere herein.

### EXAMPLE 4 (PROPHETIC)

A buffered normal saline solution including a 0.0100M acetate buffer component can be prepared by adding together and mixing the components listed in Table (8) below in the noted amounts/concentrations with enough ultrapure, deionized water to produce one liter of solution:

TABLE (8)

| Component | Amount |
|---|---|
| Sodium Chloride | 8.707 g of solid |
| Hydrochloric Acid | 0.0608 g of HCl equivalent or 1.669 mL |
| | of 1M solution |
| Sodium Acetate | 0.820 g of solid |

Bring solution volume to 1 L after dissolving the above solutes.

The buffered normal saline solution formulation of Table (8) when prepared possesses the following properties:

TABLE (9)

| | |
|---|---|
| pH | ~5.25 |
| Na⁺ | 0.159 mol/L (1.032 × the nominal of 0.154) |
| Cl⁻ | 0.151 mol/L (0.981 × the nominal of 0.154) |
| Osmolarity | 0.320 mol/L (1.036 × the nominal of 0.309) |
| Total Acetate (as acetic | 0.010 mol/L |
| acid or as acetate) | |

The buffered normal saline solution formulation of Table (8) produces an acetate-based buffer in the saline solution according to the following reaction:

NaC₂H₃O₂+HCl→Na⁺(aq)+C₂H₃O₂(aq)+H⁺(aq)+Cl⁻(aq) [symbol] Na⁺(aq)+Cl⁻(aq)+C₂H₄O₂(aq) Equation (4)

The buffered normal saline solution can then be suitable for dispensing, sterilization, and use as has been described elsewhere herein.

Note that in one embodiment, it is desirable to balance various factors in determining the amount of buffer component to add to the saline or other suitable solution. In one embodiment, these balancing factors include overall change in pH after EO sterilization, the resultant total amount of impurities remaining in the buffered saline solution after EO sterilization in view of USP guideline requirements, the length and/or number of anticipated EO sterilization cycles, and acceptable resultant osmolarity values after sterilization. In addition to these, other factors can also be taken into account when choosing the particular buffer components/sub-components and the amounts thereof.

In one embodiment, it is appreciated that the type of container in which the buffered solution is to be disposed can be considered in determining the type, amount, or concentration of buffer component to add to the saline or other solution before EO sterilization of the container. In particular, it is recognized that, in plastic syringes, at least three possible routes for undesired EO gas permeation into the container during sterilization are present: 1) through the syringe outer wall; 2) past the septum/septa of the syringe plunger tip; and 3) through the distal opening/end cap. In light of this, in one embodiment a syringe or other container in which the buffered solution is to be disposed during EO sterilization is configured to desirably minimize EO gas permeation through the container and into the buffered solution.

Specifically, in the case of a syringe, features for reducing EO gas permeation include: 1) a syringe housing outer wall including a minimum thickness of at least about 0.04 inch; 2) a plunger including three or more septa, or at least two relatively thick septa, that form a secure fit within the barrel of the syringe housing; in addition, the distance between the septa can be increased in one embodiment to lessen permeation; 3) a robust and tight-fitting end cap that covers the distal opening of the syringe so as to prevent permeation therethrough. The type of material from which the syringe or container is produced also can affect EO gas permeation into the solution contained therein. In one embodiment, the syringe includes polypropylene, polycarbonate, or other suitable plastic.

In brief, regarding the above-mentioned sterilization, in one embodiment the EO sterilization process includes exposing the buffered solution-containing containers to a warm and humid environment for a period of time to ensure a suitable termperature and humidity level, evacuating ambient air and introducing EO gas while maintaining temperature and humidity, then removing the EO gas via successive vacuum cycles. This process may be repeated one, two, or more times as needed. Autoclaving is also performed in one embodiment to sterilize the buffered solution itself. In one embodiment, the EO sterilization process occurs at a temperature of about 135 degrees F. at about 60% relative humidity at a pressure of about 28 inches of mercury with EO gas exposure to the syringes for about 2 hours or more. Pre-conditioning and post-conditioning processes are also performed.

In light of the above, it is noted that the buffer component in the buffered solution can be consumed at varying rates depending on such factors as container material type and geometry, as well as the duration, temperature, vacuum level, intensity (EO gas concentration), humidity, and number of the EO sterilization cycle(s), etc.

It is appreciated that the principles described herein relating to use of a buffer component can be extended to use on other aqueous solutions that may be used as infusates. Examples of such solutions include lidocaine, chlorhexidine, dextrose, lactated Ringer's solution, heparinized saline, total parenteral nutrition, and other medications.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A buffered infusate suitable for disposal in a container, the container to be sterilized using ethylene oxide, the buffered infusate comprising:
an aqueous solution disposed in the container, the aqueous solution suitable for infusion into a body of a patient; and
a buffer component that is added to the aqueous solution to form a buffered solution, the buffer component resisting a change in pH of the buffered solution upon exposure of the buffered solution to the ethylene oxide during the sterilization of the container.

2. The buffered infusate as defined in claim 1, wherein the buffer component includes an acid and a base in solution to counteract a change in the number of hydrogen ions in the solution.

3. The buffered infusate as defined in claim 2, wherein the acid and the base include a predetermined acid and conjugate base of the predetermined acid.

4. The buffered infusate as defined in claim 2, wherein the container includes a syringe, and wherein a cation of the base of the buffer component is commonly found in the blood of a patient.

5. The buffered infusate as defined in claim 1, wherein the buffer component includes one of a: a strong acid and a weak base; and a weak acid and a strong base.

6. The buffered infusate as defined in claim 1, wherein the aqueous solution includes a saline solution, wherein the buffer component includes acetic acid and sodium acetate, and wherein the pH of the buffered solution remains between about 4.5 and about 7 after sterilization using ethylene oxide.

7. The buffered infusate as defined in claim 1, wherein the aqueous solution includes a saline solution, wherein the buffer component includes acetic acid and sodium hydroxide, and wherein the pH of the buffered solution remains between about 4.5 and about 7 after sterilization using ethylene oxide.

8. The buffered infusate as defined in claim 1, wherein the aqueous solution includes a saline solution, wherein the buffer component includes hydrochloric acid and sodium acetate, and wherein the pH of the buffered solution remains between about 4.5 and about 7 after sterilization using ethylene oxide.

9. A method of producing a buffered infusate contained in a container, the buffered infusate suitable for withstanding the effects of sterilization of the container using ethylene oxide, the method comprising:
providing an aqueous solution, the aqueous solution suitable for infusion into a body of a patient;
adding a buffer component to the aqueous solution to form a buffered solution;
disposing the buffered solution in a container; and
sterilizing the container using ethylene oxide,
wherein the buffer component resists a change in pH of the buffered aqueous solution upon exposure of the buffered solution to the ethylene oxide during the sterilization of the container.

10. The method of producing as defined in claim 9, wherein the container includes a syringe defining a hollow portion, and wherein disposing the buffered solution further comprises filling the hollow portion of the syringe with the buffered solution and inserting a plunger into the syringe before sterilizing the syringe using ethylene oxide.

11. The method of producing as defined in claim 9, wherein providing the aqueous solution includes providing a normal saline solution, and wherein adding the buffer component includes mixing an acid, a base, and deionized water to the normal saline solution to form a buffered saline solution.

12. The method of producing as defined in claim 11, wherein the buffered saline solution includes a pH between about 4.5 and about 7 after sterilization using ethylene oxide.

13. The method of producing as defined in claim 9, further comprising heat sterilizing the buffered solution disposed in a syringe prior to sterilization of the syringe using ethylene oxide.

14. A medical device containing a buffered infusate, the medical device suitable for sterilization using ethylene oxide, the medical device comprising:
a container defining a volume in which the buffered infusate is disposed, the buffered infusate including:
an aqueous solution suitable for infusion into a body of a patient; and
a buffer component that is added to the aqueous solution to form a buffered solution, the buffer component resisting a change in pH of the buffered solution upon exposure of the buffered solution to the ethylene oxide during the sterilization of the medical device.

15. The medical device as defined in claim 14, wherein the medical device is used to infuse the infusate into the body of a patient or into another medical device associated with the patient, and wherein the buffered solution is exposed to the ethylene oxide via gas permeation into the medical device.

16. The medical device as defined in claim 14, wherein the medical device includes a syringe, the syringe including a plunger with a plunger tip, the plunger tip defining a plurality of septa.

17. The medical device as defined in claim 16, wherein the buffer component includes an acid and a base including a sodium cation.

18. The medical device as defined in claim 14, wherein the sterilization includes insertion of the medical device into an ethylene oxide gas environment for at least two hours at a temperature of at about 135 degrees F. at about 60% relative humidity.

19. The medical device as defined in claim 14, wherein the aqueous solution includes at least one of saline, dextrose, chlorhexidine, lidocaine, lactated Ringer's solution, heparinized saline, and total parenteral nutrition.

20. The medical device as defined in claim 14, wherein the buffer component includes one of citric acid, lactic acid, benzoic acid, sorbic acid, maleic acid, phosphoric acid, formic acid, ascorbic acid, and carbonic acid together the corresponding conjugate base.

21. A buffered saline infusate suitable for disposal in a syringe, the syringe to be sterilized using ethylene oxide, the buffered saline infusate comprising:
an aqueous saline solution suitable for infusion into a body of a patient, the saline solution disposed in the syringe; and
a buffer component included with the saline solution to form a buffered saline solution, the buffer component including acetic acid and acetate so as to resist a change in pH of the buffered saline solution upon exposure of the buffered saline solution to the ethylene oxide during the sterilization of the syringe.

22. The infusate as defined in claim 21, wherein the saline solution is a normal saline solution including 0.9% saline by weight in an aqueous solution.

23. The infusate as defined in claim 22, wherein about 1 liter of the buffered solution includes in water solution about 8.7 grams sodium chloride, about 0.68 grams sodium acetate, and about 1.67 mL of 1 Molar acetic acid.
